(19) United States
(12) Patent Application Publication
(10) Pub. No.: US 2004/0132780 A1
(43) Pub. Date: Jul. 8, 2004

(54) Method and compositions for treating migraines

(76) Inventors: Christopher P. Allen, Doylestown, PA (US); Phyllis W. Stone, Somerset, NJ (US); Sean Harper, Gwynedd Valley, PA (US)

Correspondence Address: MERCK AND CO INC, P O BOX 2000, RAHWAY, NJ, 070650907

(21) Appl. No.: 10/476,753

(22) PCT Filed: Apr. 30, 2002
(86) PCT No.: PCT/US02/13750

(30) Foreign Application Priority Data
May 4, 2001 (US) 60/288623

(51) Int. Cl. A61K 31/444

(52) U.S. Cl. 514/334

(57) ABSTRACT

The present invention relates to a method for treating or preventing migraines in a mammalian patient in need of such treatment or prevention comprising administering to said patient a compound of Formula A or a pharmaceutically salt, hydrate or N-oxide thereof, in an amount that is effective to treat or prevent migraines.

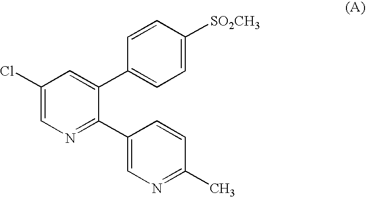

### BACKGROUND OF THE INVENTION

Migraines are recurrent, often familial, symptom complexes of periodic attacks of vascular headache. The condition is characterized by intermittent attacks of headache, preceded by an aura in approximately 15% of patients. The headache is often accompanied by associated symptoms, most commonly nausea, vomiting, photophobia and phonophobia. Migraines affect approximately 17% of adult women and 6% of adult men (Stewart et al., Neurology, 1994, 44 (suppl. 4), 517-523).

Cyclooxygenase (COX), also known as prostaglandin H synthase, is an enzyme implicated in the mediation of pain, fever and inflammation. It catalyzes the oxidative conversion of arachidonic acid into prostaglandin H₂, a key intermediate in the biosynthetic pathway of prostaglandins, prostacyclins and thromboxanes, which in turn mediate a variety of physiological effects both beneficial and pathological.

Recently it was discovered that two COX isoforms exist: COX-1, expressed constitutively in many tissues, and COX-2, an induced isoform having elevated levels of expression in inflamed tissues. COX-1 is thought to be involved in ongoing “housekeeping” functions, for example, gastric cytoprotection, while COX-2 is implicated in the pathological effects mentioned above.

Current cyclooxygenase inhibitors such as aspirin, ibuprofen and indomethacin, used as non-steroidal anti-inflammatory drugs (NSAIDs), inhibit both COX-1 and COX-2 and have associated side effects, such as gastrotoxicity, which may be manifested as ulcer formation. COX-2 selective inhibitors act as effective NSAIDs without substantial gastrotoxic side effects. For purposes of this disclosure only, a COX-2 selective inhibitor is defined as a COX inhibitor having a selectivity for the COX-2 isoform relative to the COX-1 isoform.

### SUMMARY OF THE INVENTION

The present invention relates to a method for treating or preventing migraines in a mammalian patient in need of such treatment or prevention comprising administering to said patient a compound of Formula A:

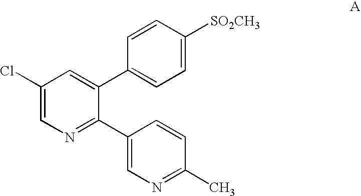

or a pharmaceutically acceptable salt, hydrate or N-oxide thereof, in an amount that is effective to treat or prevent migraines.

### DETAILED DESCRIPTION

The present invention encompasses a method for treating or preventing migraines in a mammalian patient in need of such treatment or prevention comprising administering to said patient a compound of Formula A:

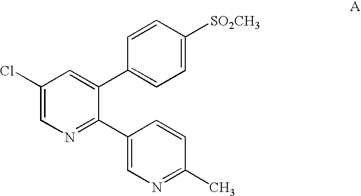

in an amount that is effective to treat or prevent migraines.

The compound of Formula A, which has the generic name etoricoxib, is a selective inhibitor of cyclooxygenase-2. Etoricoxib is disclosed as Example 23 in U.S. Pat. No. 5,861,419, issued on Jan. 19, 1999, which is hereby incorporated by reference in its entirety.

In an embodiment of the invention the compound of Formula A is administered at a dose ranging from about 10 mg to about 200 mg. In another embodiment of the invention the mammalian patient is human.

Another embodiment of the invention encompasses a method for treating migraines in a mammalian patient in need of such treatment comprising administering to said patient a compound of Formula A:

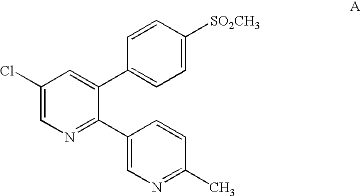

or a pharmaceutically acceptable salt, hydrate or N-oxide thereof, in an amount that is effective to treat migraines.

For purposes of this specification, treating migraines means relieving both the headache and the consequent associated symptoms of migraine. Treating migraines is synonymous with the acute treatment of migraines.

Another embodiment of the invention encompasses a method for preventing migraines in a mammalian patient in need of such prevention comprising administering to said patient a compound of Formula A:

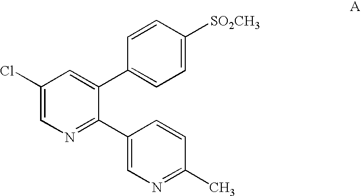

or a pharmaceutically acceptable salt, hydrate or N-oxide thereof, in an amount that is effective to prevent migraines.

For purposes of this specification, prevention of migraines means reducing the severity, the frequency or both the severity and frequency of migraine attacks. Preventing migraines is synonymous with migraine prophylaxis or the chronic treatment of migraines.

For purposes of this specification, migraine is meant to include migraine without aura, migraine with aura, migraine with typical aura, migraine with prolonged aura, familial hemiplegic migraine, basilar migraine, migraine aura without headache, migraine with acute onset aura, ophthalmoplegic migraine, retinal migraine, childhood periodic syndromes that may be precursors to or associated with migraine, benign paroxysmal vertigo of childhood, alternating hemiplegia of childhood, status migrainosus and migrainous infarction. Reference is made to the following: Headache Classification Committee of the International Headache Society: Classification ad diagnostic criteria for headache disorders, cranial neuralgias and facial pain. Cephalalgia. 1988;8(suppl 7):1-96, which is hereby incorporated by reference in its entirety.

Etoricoxib has a shorter time to maximum concentration and longer half-life as compared to traditional NSAIDs such as naproxen and will therefore have greater efficacy in the acute treatment of migraine. Etoricoxib is also better suited than traditional NSAIDs for chronic administration.

For purpose of this specification, an amount that is effective to treat or prevent migraines is that amount that will relieve the subject being treated of the symptoms of the migraine attack and the specific dose level and frequency of dosage may vary and will depend upon a variety of factors including the activity of the specific compounds used in combination, the metabolic stability and length of action of the compounds, the age, body weight, general health, sex diet, mode and time of administration, rate of excretion, the severity of the particular condition and the host undergoing therapy. However, dosage levels of etoricoxib on the order of about 0.01 mg/kg to about 100 mg/kg of body weight per day, typically about 0.1 to about 10 mg/kg, more particularly about 0.2 to about 5 mg/kg and especially about 0.14 to about 3 mg/kg per day are useful in the novel method of treatment. For the treatment of a migraine attack, the active ingredient may be administered orally, topically, parenterally, by inhalation, spray, rectally or intravaginally in formulations containing pharmaceutically acceptable carriers.

The term parenteral as used herein includes subcutaneous injections, intravenous, intramuscular, intracisternal injection or infusion techniques.

Etoricoxib may be in a form suitable for oral use, for example, tablets, troches, lozenges, aqueous or oily suspensions, dispersible powders or granules, emulsions, hard or soft capsules, solutions, syrups and elixirs. Compositions intended for oral use may be prepared according to any method known to the art for the manufacture of pharmaceutical compositions and typically such compositions contain one or more agents selected from the group consisting of sweetening agents, flavoring agents, coloring agents and preservatives in order to provide pharmaceutically elegant and palatable preparations. These excipients may be for example, diluents such as lactose, calcium carbonate, sodium carbonate, calcium phosphate or sodium phosphate; granulating and disintegrating agents, for example, corn starch or alginic acid; binding agents, for example starch, gelatin or acacia, and lubricating agents, for example, magnesium stearate, stearic acid or talc.

The tablets may be uncoated or they may be coated. Coating can be included to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or miscible solvents such as propylene glycol, PEGs and ethanol, or an oil medium, for example peanut oil, liquid paraffin or olive oil.

Aqueous suspensions contain the active material in admixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, tragacanth and acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxy-ethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain demulcents, preservatives, flavorants and coloring agents.

The pharmaceutical compositions may be in the form of a sterile injectable aqueous or oleaginous suspension. This suspension may be formulated according to the known art using those suitable dispersing or wetting agents and suspending agents which have been mentioned above.

Injectable compositions are typically in the form of sterile solutions or suspensions, which include the active ingredient in a parenterally acceptable diluent. Among these are sterile water, dextrose 5% in water (D5W), Ringer's solution and isotonic saline, as well as mixtures thereof. Cosolvents such as ethanol, propylene glycol or polyethylene glycols may also be used. Sterile, injectable oil is occasionally employed as a solvent or suspending medium in intramuscular preparations. A representative example is peanut oil. In addition, fatty acids such as oleic acid, preservatives, buffers and local anesthetics find use in the preparation of intramuscular injectables.

The combination of active ingredients may also be administered rectally or intravaginally as suppositories. These can be prepared by mixing the drug with a suitable non-irritating excipient which is solid at ordinary room temperature but molten at normal or elevated body temperature. Examples of such materials include cocoa butter and polyethylene glycols.

For topical use, creams, ointments, gels, solutions, suspensions and the like containing the compound are employed. (For purposes of this application, topical application includes mouth washes and gargles, as well as transdermal applications.) Topical formulations are comprised of a pharmaceutical carrier, which may include, e.g., cosolvents, emulsifiers, penetration enhancers, preservatives or emollients.

The active ingredient is combined with the carrier to produce the dosage form. For example, a formulation intended for oral administration may contain from as low as about 0.7 mg of etoricoxib to as high as about 7 g of etoricoxib per dose, compounded with an appropriate and convenient amount of carrier material which may vary from about 5 to about 95 percent of the total composition. A preferred pharmaceutical composition contains from about 10 mg to about 200 mg of etoricoxib or a salt thereof.

Etoricoxib may also be administered in combination with other agents for the treatment or prevention of migraines. Such administration may either be in unit dosage form or concomitantly. All conventional anti-migraine agents are used in conjunction with the etoricoxib at conventional doses that are determined by the skilled clinician. These compounds are known and normal daily dosages are well established. Typically, the individual daily dosages for these combinations may range from about one-fifth of the minimally recommended clinical dosages to the maximum recommended levels for the entities when they are given alone. Precise dosages are left to the discretion of the physician Thus, in further aspects, the invention encompasses pharmaceutical compositions for treating or preventing migraines comprising etoricoxib and one or more agents selected from the group consisting of: rofecoxib, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide and prochlorperazine, in combination with a pharmaceutically acceptable carrier. In a preferred embodiment, the invention encompasses a pharmaceutical composition comprising etoricoxib and metoclopramide, in combination with a pharmaceutically acceptable carrier.

In another aspect, the invention encompasses a method for treating or preventing migraines in a mammalian patient in need of such treatment or prevention comprising administering to said patient a compound of Formula A:

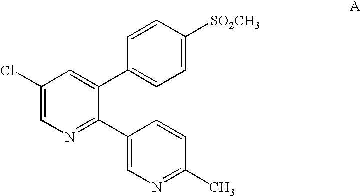

or a pharmaceutically salt, hydrate or N-oxide thereof, in combination with one or more agents selected from the group consisting of: rofecoxib, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-α-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide and prochlorperazine, in amounts that are effective to treat or prevent migraines. A preferred agent is metoclopramide.

What is claimed is:

1. A method for treating or preventing migraines in a mammalian patient in need of such treatment or prevention comprising administering to said patient a compound of Formula A: 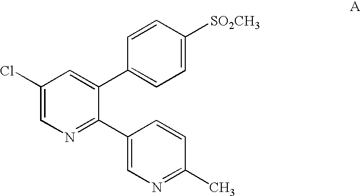
or a pharmaceutically acceptable salt, hydrate or N-oxide thereof, in an amount that is effective to treat or prevent migraines.

2. The method according to claim 1 wherein the compound of Formula A is administered at a dose ranging from about 10 to about 200 mg.

3. The method according to claim 1 wherein the mammalian patient is human.

4. The method for treating migraines in a mammalian patient in need of such treatment comprising administering to said patient a compound of Formula A: 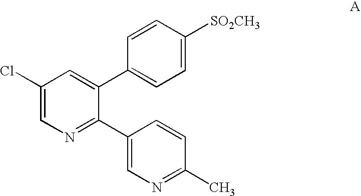
or a pharmaceutically acceptable salt, hydrate or N-oxide thereof, in an amount that is effective to treat migraines in accordance with claim 1.

5. The method for preventing migraines in a mammalian patient in need of such prevention comprising administering to said patient a compound of Formula A: 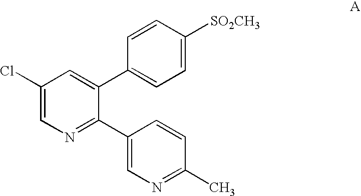
or a pharmaceutically acceptable salt, hydrate or N-oxide thereof, in an amount that is effective to prevent migraines in accordance with claim 1.

6. The method according to claim 1 further comprising administering to said patient one or more agents selected from the group consisting of: rofecoxib, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide and prochlorperazine, in an amount that is effective to treat or prevent migraines.

7. The method according to claim 6 wherein agent is metoclopramide.

8. A pharmaceutical composition comprising a compound of Formula A: 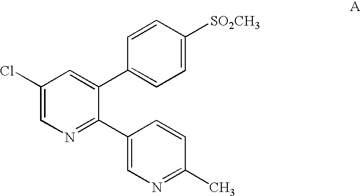
and one or more agents selected from the group consisting of: rofecoxib, indomethacin, sulindac, etodolac, mefenamic acid, meclofenamic acid, flufenamic acid, tolfenamic acid, etofenamic acid, tolmetin, ketorolac, diclofenac, ibuprofen, naproxen, fenoprofen, ketoprofen, oxaprozin, piroxicam, meloxicam, tenoxicam, lornoxicam, cinnoxicam, sudoxicam, tenoxicam, phenylbutazone, oxyphenbutazone, apazone, azapropazone, nimesulide, diflunisal, nabumetone, aspirin, sodium salicylate, choline, magnesium trisalicylate, salsalate, diflunisal, salicylsalicyclic acid, sulfasalazine olsalazine, ergotamine, ergonovine, ergonovine, mesylates, ergometrine, methylergonovine, methylsergide, metergoline, ergoloid mesylate, dihydroergotamine, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostine, bromocriptine, amitriptyline, methysergide, propranolol, valproate, verapamil, metoclopramide and prochorperazine, in combination with a pharmaceutically acceptable carrier.

9. The pharmaceutical composition according to claim 8 comprising a compound of Formula A: 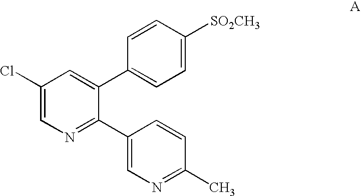
and metoclopramide in combination with a pharmaceutically acceptable carrier.
